(19) United States
(12) Patent Application Publication
(10) Pub. No.: US 2024/0052502 A1
(43) Pub. Date: Feb. 15, 2024

(54) PLASMA ASSISTED ELECTROCATALYTIC CONVERSION

(71) Applicants: NewSouth Innovations Pty Limited, Sydney, New South Wales (AU); The University of Sydney, Sydney, New South Wales (AU)

(72) Inventors: Emma Lovell, Sydney, New South Wales (AU); Rose Amal, Sydney, New South Wales (AU); Rahman Daiyan, Sydney, New South Wales (AU); Rouhollah Jalili, Sydney, New South Wales (AU); Patrick J. Cullen, Sydney, New South Wales (AU); Hassan Masood, Sydney, New South Wales (AU); Jing Sun, Sydney, New South Wales (AU); David Alam, Sydney, New South Wales (AU); Tianqi Zhang, Sydney, New South Wales (AU); Renwu Zhou, Sydney, New South Wales (AU)

(21) Appl. No.: 18/248,167

(22) PCT Filed: Oct. 7, 2021
(86) PCT No.: PCT/AU2021/051172

§ 371 Date: Apr. 6, 2023

(30) Foreign Application Priority Data
Oct. 7, 2020 (AU) 2020903637

(51) Int. Cl. C25B 1/27; C25B 1/50; C25B 15/027; C25B 15/031; C25B 11/031; C25B 11/061; C25B 11/075

(52) U.S. Cl. CPC C25B 1/27; C25B 1/50; C25B 15/027; C25B 15/031; C25B 11/031; C25B 11/061; C25B 11/075

(57) ABSTRACT

A method of reducing a gaseous compound, for example, nitrogen or carbon dioxide, the method comprising the steps of subjecting the gaseous compound to plasma forming conditions to form a plasma; contacting the plasma with water or an electrolyte at a plasma-water or electrolyte-water interface, thereby to provide a dissolved plasma derived species; and electrocatalytically reducing said dissolved plasma derived species to provide a reduced compound. The plasma may for example be generated by a combination of glow discharge and spark discharge in a configuration of a pin-to-liquid with no enclosure, pin-to-liquid with nozzle enclosure, or a pin-to-liquid with a column bubbler enclosure. A catalyst, such as transition metal, maybe added, advantageously in the form of a nano structured catalyst.

### TECHNICAL FIELD

The invention relates to a new, hybrid technology for the production of reduced species, such as ammonia via clean and renewable sources. The technology is based on the coupling of plasma-assisted activation of gas and electrocatalytic conversation of relevant plasma species. The invention also relates to apparatus and catalysts suitable for use in the system.

### BACKGROUND

The transformation of atmospheric nitrogen (N₂) to ammonia (NH₃) is essential for many eco-systems and industrial processes. Currently, only bacteria and some plants can synthesize ammonia from air and water at ambient conditions via nitrogen fixation processes.

Ammonia is an extremely valuable global commodity at present and seems likely to play a significant role not only in manufacturing but also in energy production and storage in the near future.

Globally, approximately $60 billion worth of ammonia is produced every year for utilisation, mostly in the form of fertilizers. It is estimated that at least half the nitrogen in the human body today comes from a synthetic ammonia plant. Recently, ammonia has been gaining increasing attention as a hydrogen carrier for the hydrogen economy. Ammonia stores almost twice as much energy as liquid hydrogen and is easier to ship and distribute for export purposes. Thus, the global ammonia market has significant potential for expansion in upcoming years.

Commercially, ammonia production has remained essentially unchanged since World War I. The Haber-Bosch process was developed in the early 20^{th} Century and is very well known. The conversion typically occurs at high pressures (150-250 atmospheres) and high temperatures (400-500° C.). Additionally, for this process, relatively high purity hydrogen (from steam reforming of methane) and nitrogen (from air separation) feeds are required. Because of this, the process consumes a significant amount of energy and is fundamentally incompatible with small scale, delocalised ammonia production as well as making it unfeasible to accommodate intermittent and diffusive renewable energy.

In recent years, the production of ammonia through electrocatalytic nitrogen reduction reaction (eNRR) has gained increasing attention. Electrocatalytic NRR has significant benefits over the Haber-Bosch process including operating at mild conditions (ambient temperature and pressures), being fundamentally compatible with renewable energy, well-suited to delocalized production and distribution as well as not requiring any hydrogen feed (with hydrogen coming from water/the electrolyte).

Despite these benefits, electrocatalytic NRR remains significantly hindered with low yields of ammonia and the difficulties in achieving high Faradaic efficiencies at low overpotentials. Specifically, the use of eNRR is intrinsically limited due to the highly unreactive nature of N₂ and its low solubility in water. Moreover, the eNRR is hampered by the competition with hydrogen evolution reaction (HER), as hydrogen generation usually occurs at a lower overpotential than the eNRR. Consequently, the eNRR remains significantly hindered by low ammonia production rates (typically 10⁻⁹ to 10⁻¹⁰ mol cm⁻² s⁻¹) making reliable detection troublesome and, with few exceptions, very low Faradaic efficiencies, below 1%.

To date, some of the highest yields achieved electrocatalytically have been in the order of ˜5 μg/cm²/h, bringing questions about potential scalability in the future.

A promising approach to overcoming the limitations of eNRR is converting N2 into a more reactive intermediary form. In this context, lithium redox intermediary NRR has attracted some recent attention to achieve higher rates and current densities than eNRR. However, the significant overpotential of 3V minimum for the Li-NRR makes this process inherently energy-intensive. Moreover, system stability, the need for ultra-dry and oxygen-free organic solvents and their decomposition at the anode, high pressures (˜50 bar) and hydrogen feed and lithium metal requirements are additional drawbacks of this pathway.

Nitrite and nitrates (NO₃⁻ and NO₂⁻, respectively), also referred to collectively when mixed as NOₓ, are highly soluble and much more easily reduced to ammonia than N₂ and benefits from already known chemistry. As such, the generation and exploitation of NOₓ as an intermediary to overcome the limitations of N₂ conversion presents a novel solution to these limitations. However, in industry, nitrites and nitrates are produced from ammonia via the Ostwald process; thus, their direct use as precursors for ammonia production is unfeasible. Additionally, nitrates/nitrites have limited stability in water hence direct production and on-spot utilization is essential. Consequently, the production of NOₓ for immediate consumption to produce ammonia is of critical industrial importance.

A number of electrocatalytic approaches to the conversion of inert molecules (such as nitrogen) into valuable products have been attempted but these have been significantly hindered from both a conversion and selectivity point of view. In particular, the solubility and activation of these molecules limits conversion and applications in conventional electrocatalysis.

Prior art methods have been attempted using plasma but these invariably the plasmas are generated under oxygen-free conditions, that is the prior art methods have sought to avoid the generation of NOₓ species by using dry nitrogen or nitrogen in combination with a gas such as helium. Other than eliminating NOₓ generation possibilities, the use of helium also leads to variation in plasma energetics as high energy electrons can be obtained at lower potentials compared to nitrogen and oxygen mixtures.

It is an object of the present invention to overcome or ameliorate at least one of the disadvantages of the prior art, or to provide a useful alternative.

### SUMMARY

The invention is a hybrid plasma-electrocatalytic system which activates an input feed gas, in particular nitrogen, which may be in the form of air via plasma formation at the interface of the gas and a liquid (water or electrolyte). The activated NOₓ species are subsequently dissolved in the liquid, where they are then converted electrocatalytically into ammonia.

In a broad aspect, the invention provides a method of reducing a gaseous compound comprising the steps of:
subjecting the gaseous compounds to conditions enable formation of plasma;
contacting the plasma with water or an electrolyte at a plasma-water or electrolyte-water interface, thereby to provide a dissolved plasma derived species; and
electrocatalytically reducing said dissolved plasma derived species to provide a reduced compound.

The gaseous compound may be nitrogen-containing (e.g. air), in which case the reduced compound is ammonia. Alternatively, the gaseous compound is an oxygen containing compound, for example, carbon dioxide (which may produce carbon monoxide and formates etc. as the reduced compound) or a compound mixed with oxygen.

Preferably, the plasma is generated by a combination of glow discharge and spark discharge, albeit either glow or spark discharge could also be viably utilized independently. The plasma electrode embodiments are pin-to-liquid with no enclosure, nozzle enclosure, and bubble column enclosure. The latter is preferred as it provides plasma-water or electrolyte water interface at the interface of a bubble of gas in the water or electrolyte. Preferably, one or both plasma electrodes are covered with a dielectric barrier. In one embodiment the high-voltage electrode is partially covered to offer a combination of glow and spark discharge. Preferably, the ground electrode is of similar design as high voltage electrode and placed inside the liquid. In other configurations dielectric barrier separates the ground and the liquid.

Preferably, in configurations exhibiting underwater plasma bubbles, residence time of bubbles in the liquid is facilitated by Raschig rings.

Preferably, the glow discharge region inside plasma bubble column is packed with metal oxides in form of nanoparticles or monolith with tuneable void fraction and bed height.

Preferably, the gaseous compound is provided at controlled humidity. Also, it is preferred if the water or electrolyte is provided at a controlled temperature. It is also preferred that the water or electrolyte is provided at a controlled pH (both alkaline and acidic). Gas humidity, water temperature and pH can be controlled by conventional method in the art, such as desiccants/bubblers, heaters/coolers and buffers respectively.

The plasma activation and electrolysis can be carried out at any pH range from pH 0 to pH 14 (i.e. for example, in the range of pH's between that of a 1M H⁺ solution and a 1M OH⁻ solution.

Preferably, the electrocatalytic reduction is facilitated by a transition metal catalyst. Particularly suited transition metals include copper, nickel, tin, bismuth, cobalt, titanium or iron, or the oxides of said transition metals and mixtures thereof.

Preferably the transition metal catalyst is in the form of a foil, a foam, a nanostructured catalyst, a nanoparticulate catalyst or a single atom metal on doped-carbon catalyst. In a preferred form, the catalyst is in the form of a foam with deposits (particularly nanodeposits), such as a metal foam supporting nanowires. Highly preferred are copper catalysts in the form uniformly dispersed thin nanowires on a copper foam. Most preferably, the catalyst is in the form of a metal foam supporting nanowires, such as a copper foam supporting copper nanowires with surface defects.

The term “nanostructured” as herein defined refers to structures that have a feature having at least one dimension on the nanoscale, that is, between 0.1 nm and 1000 nm, preferably between 0.1 nm and 500 nm.

The term “nanowire” as herein defined refers to an elongate wire having a diameter on the nanoscale, that is, between 0.1 nm and 1000 nm, preferably between 0.1 nm and 500 nm. Preferably, the nanowire also has a high length-width ratio of the order of 100 or more or even 1000 or more.

It is preferred that, where present, the catalyst is located in the reaction system in a region adjacent the region of the spark discharge and/or glow discharge.

Preferably the dissolved plasma species is reduced without isolation. In one embodiment, the dissolved plasma species are reduced in the vessel in which plasma is generated. In an alternative embodiment, the dissolved plasma species are stored in a reservoir prior to electrocatalytic reduction.

In a preferred aspect, the invention provides a method of reducing nitrogen gas to produce ammonia, the method comprising the steps of:
subjecting the nitrogen-containing gas to plasma forming conditions to form a nitrogen-containing plasma;
contacting the nitrogen-containing plasma with water or an electrolyte at a plasma-water or electrolyte-water interface, thereby to provide dissolved NOₓ species;
storage of these dissolved species in a reservoir with potential dosing to vary conductivity and pH; and electrocatalytically reducing said NOₓ to provide ammonia.

The nitrogen containing gas may be pure nitrogen or substantially pure nitrogen, or it may be in admixture with other species. For preference, the nitrogen containing gas further comprises oxygen.

The nitrogen:oxygen ratio may be any ratio between 1:99 and 99:1 wt:wt. In one particular aspect, the nitrogen containing gas is air.

Preferably, the plasma is generated by a combination of glow discharge and spark discharge. The configuration of the plasma electrodes can be a pin-to-liquid with no enclosure, pin-to-liquid with nozzle enclosure, or a pin-to-liquid with a column bubbler enclosure. Most preferably, the plasma is generated by a combination of glow discharge and spark discharge.

Preferably, the plasma-water or electrolyte water interface is at the interface of a bubble of gas in the water or electrolyte.

The gaseous compound may be provided at any relative humidity (i.e. 0 to 100%). In some embodiments, such as carrying out the reduction of nitrogen, a dry gas may be preferred. In some embodiments the gaseous compound is provided at a relative humidity of 20-80%. Preferably, the water or electrolyte temperature is between 20 and 80° C.

In certain preferred embodiments, the electrolyte is aqueous H₂SO₄ or HCl. Other acidic electrolytes may also be used. In other preferred embodiments, the electrode is a basic species, such as an aqueous solution of hydroxide salt, e.g. KOH or NaOH. In still other preferred embodiments the electrolyte is pure water or an aqueous salt solution (such as, for example KCl or NaCl)

Preferably the dissolved NOₓ species are NO₂_{^{−}} or NO₃_{^{−}} or a mixture comprising at least both NO₂_{^{−}} and NO₃_{^{−}}.

In one embodiment, the dissolved NOₓ species are moved from a generation vessel to a reservoir prior to electrocatalytic reduction. In another embodiment, the dissolved NOₓ species are electrocatalytically reduced in the vessel in which they are generated.

In an alternative aspect, the invention provides a method of reducing carbon dioxide gas comprising the steps of:
subjecting the carbon dioxide gas to plasma forming conditions to form activated species;
contacting the activated species with water or an electrolyte at a plasma-water or electrolyte-water interface, thereby to facilitate dissolution of species; and
electrocatalytically reducing said dissolved species to provide one or more reduced compounds selected from CO, syngas or formate.

The gaseous compound contains an oxygen source. In some embodiments that is covalently bound to the reduced species, such as in the case of CO₂, where the carbon is bound to the oxygen, or the oxygen source is in the form of gas mixed with the reducible species or co-fed to a plasma forming gas stream, for example, where a nitrogen gas is being reduced, oxygen may be added to the plasma forming feed in a predetermined, controlled amount, or the feed gas to the plasma may be air.

In another aspect, the invention provides apparatus for reducing a gas comprising
i) a feed line to feed the gas to a glow/spark plasma discharge (pin-to-liquid with no enclosure, pin-to-liquid with nozzle enclosure, or a pin-to-liquid with a column bubbler enclosure) located at or below a liquid level in a reaction vessel;
ii) the pin-to-liquid with no enclosure, pin-to-liquid with nozzle enclosure, or a pin-to-liquid with a column bubbler enclosure having a housing configured to generate bubbles in the liquid in a reaction vessel when in use; and
iii) a feed line to transport dissolved plasma species from the reaction vessel to an electrocatalytic reduction chamber.

In yet another aspect, the invention provides apparatus for reducing a gas comprising
i) a feed line to feed the gas to a glow/spark plasma discharge (pin-to-liquid with no enclosure, pin-to-liquid with nozzle enclosure, or a pin-to-liquid with a column bubbler enclosure) located at or below a liquid level in a reaction vessel;
ii) the pin-to-liquid with no enclosure, pin-to-liquid with nozzle enclosure, or a pin-to-liquid with a column bubbler enclosure having a housing configured to generate bubbles in the liquid in a reaction vessel when in use;
iii) a fluid line to transport dissolved plasma reaction products from the reaction vessel to a reservoir; and
iv) a feed line to transport dissolved plasma species from the reservoir to an electrocatalytic reduction chamber.

The invention also provides a catalyst comprising a transition metal catalyst in the form of a nanowire. Preferably the nanowire is supported on a transition metal foam. Most preferably the catalyst is a copper nanowire supported by a copper foam.

### BRIEF DESCRIPTION OF THE DRAWINGS

FIG. 1 shows Schematics showing different configurations of plasma-driven gas activation including (a) pin-to-liquid with no enclosure, (b) pin-to-liquid with nozzle enclosure and (c) pin-to-liquid with a column bubbler enclosure.

FIG. 2 shows impact of voltage and frequency on the total production of NOₓ for the pin-in-nozzle design.

FIG. 3 shows Plasma reactor design and busting energy efficiency. (A) Energy efficiency and NOₓ production rate with schematics representing plasma bubble column reactors design configurations: single reactor glow discharge (SRGD); single reactor spark discharge (SRSD); single reactor glow and spark discharge; double reactor glow and spark discharge (DRGSD); and DRGSD with Raschig rings. (B) Photo and (C) schematic showing the combined double reactor glow and spark discharge with Raschig rings, (D) plasma bubble representative photo, (E) and (F) showing optical emission spectra (OES) for the glow and spark discharges, respectively.

FIG. 4 shows Ammonia production rate and the corresponding faradaic efficiency for plasma-activated water (PAW) compared to salt solution with similar NOₓ concentrations

FIG. 5 shows Electrochemical optimization to increase the production rate and Faradic efficiency on a small scale. (A) Digital photo showing in the plasma discharge in solution. Schematics outlining the (B) plasma activation of air and water, producing NOₓ dissolved in the electrolyte as an intermediary of the electrochemical synthesis of ammonia in an H-cell. (C-D) Scanning electron microscopy (SEM) of as-prepared Cu-NW catalyst. (E) Linear sweep voltammetry (scan rate of 5 mV·s⁻¹) of Cu-NW and the background electrolyte (10 mM H₂SO₄). (F) NH₃ production rate and Faradaic efficiency as a function of applied potential for Cu-NW electrode. Please note, no hydrogen was detected via GC connected to the cell. (G) The time-dependent concentration of NO₃_{^{−}}, NO₂_{^{−}}, and ammonium during 2.5 h electrolysis at −0.5 V vs RHE with Cu-NW. Note: all testing was conducted in a custom-designed H-cell using a 1 cm×1 cm electrode and plasma-activated electrolyte without iR correction.

FIG. 6 shows Schematics of plasma-electrocatalytic NRR systems showing (a) H-cell integration as well as (b) flow cell system both using plasma bubbler.

FIG. 7 shows Optimization of the flow-through hybrid system for high energy efficiency and yield. (A) Schematic of the flow-through system with the plasma-bubbler having a liquid outlet leading to the flow-through electrolyser to convert the NOx to ammonia. (B) Reported ammonia production rate and energy consumption for other NRR systems in the literature (eNRR), Li-intermediary NRR, and plasma-assisted NRR are shown. Data for the hybrid system of the present invention are shown for comparison. (C) Cell potential and Faradaic efficiency of ammonia synthesis over 8 hours at 30 mA/cm₂. (D) NH₃ production rate and current density as a function of cell voltage.

FIG. 8 shows Photographs of plasma bubble column reactors design configurations: (A) single reactor glow discharge (SRGD); (B) single reactor spark discharge (SRSD); (C) single reactor glow and spark discharge (SRGSD); (D) double reactor glow and spark discharge (DRGSD); and (E) DRGSD with Raschig rings. Schematic diagrams are shown below the respective photographs

FIG. 9 shows species generated in spark and glow phases and at the water interface.

FIG. 10 shows Plasma catalysis NOx synthesis rate by using TiO₂ 7050 catalyst with different weight concentrations of graphene oxide binder.

FIG. 11 shows XPS of the Cu2p of the electrodes: (a) Cu Foam; (b) CuO NW; (c) Cu NW (after electroreduction) and (d) Cu NW (after used for reaction).

FIG. 12 shows XPS of the N1s of the Cu-NW electrodes A) before and B) after the electrocatalytic tests.

FIG. 13 shows Time-dependent concentration of plasma generated NO₃— and NO₂— in the H-cell containing 100 ml of water.

FIG. 14 shows Optimization of the electrolyte via adjusting the pH of electrolyte using sulfuric acid. (A) Ammonia production rates and the corresponding Faradaic Efficiencies in different sulfuric acid concentrations at −0.5V vs RHE for 15 min and using Cu foil (1 cm×1 cm) as the catalyst; (B) Representative linear sweep voltammetry (LSV, 0V to −1V) of electrolyte containing various concentrations of sulfuric acid. 0.5M Na₂SO₄ was added to adjust conductivity when there was no acid in the electrolyte.

FIG. 15 shows nitrite and nitrate salts potentials study. (A) ammonia production rate and the corresponding FE of 1 mM NaNO₂ solution under different potentials from −0.2V to −0.6V; (B) ammonia production rate and the corresponding FE of 1 mM KNO₃ solution under different potentials from −0.2V to −0.6V.

FIG. 16 shows LSV curves (scan rate of 5 mV·s−1) of 1 mM NaNO₂ and 1 mM KNO₃ solutions from 0V to −0.8V

FIG. 17 shows NOₓ (nitrite and nitrate) concentration study to increase rate and FE. Ammonia production rate and the corresponding FE in different concentrations of NaNO2 and KNO3 solutions under −0.5V vs RHE for 15 minutes.

FIG. 18 shows ammonia production rate as a function of applied potential.

FIG. 19 shows (A) LSV curves of copper catalysts with different porosity (Cu foil, Cu foam and Cu NWs) in PAW electrolyte; (B) ammonia production rates and FE using these catalysts.

FIG. 20 shows Electrochemically active surface area (ECSA) comparison of various forms of copper catalysts (Cu foil, Cu foam and Cu NWs in 0.5M Na₂SO₄ solution) used in this study.

FIG. 21 shows the electrocatalytic reduction of nitrate to ammonia catalysed by single atom Ni sites.

FIG. 22 shows the electrocatalytic reduction of nitrate to ammonia catalysed by single atom Cu sites.

FIG. 23 shows Time-dependent concentration of NO₃—, NO₂—, and ammonium during 2.5 h electrolysis at −0.5 V vs RHE with Cu NWs with sampling of nitrite, nitrate and ammonia along with the chronoamperometric i-t curve.

FIG. 24 shows a typical 1D 1H spectrum obtained with NMR analysis on liquid aliquots (A) taken from plasma activated water (PAW); (B) taken after 2.5 h of electroreduction of PAW.

### DESCRIPTION

The present invention relates to a new, hybrid technology for the production of a reduced gaseous species (such as ammonia) via clean and renewable sources. The technology is based on the coupling between two fundamental aspects: plasma-assisted activation of gas; and electrocatalytic conversation of relevant plasma species to the reduced gaseous species.

Gaseous species, for example, ground-state nitrogen molecules, exhibit high ionization potential. This is intrinsically non-reactive for thermodynamic standpoint, but plasma activation can provide avenues for the conversion of highly stable nitrogen molecules into easier-to-breakdown species. These species can then be more efficiently converted into ammonia electrochemically. The hybrid system of the present invention can operate under ambient conditions, with water and air being reactants.

Further, the ammonia produced is in aqueous phase, thus requiring no further pre-treatment stages for application areas such as direct use as fertilizer and in the textile and explosives industries. The present invention is a hybrid plasma-electrocatalytic system which activates an input feed gas to form a plasma at the liquid/gas interface of the reactant gas within the liquid (typically water/electrolyte). The resulting activated species are dissolved in the liquid, and subsequently converted into valuable chemicals by means of electrocatalysis. The invention relates to the method, apparatus and also to specific features of the system, in particular features such as the catalyst design.

This present invention can be used to convert a variety of reducible gases into reduced species but in general, it will be discussed herein with reference to the conversion of nitrogen (either as supplied nitrogen or air) and water to ammonia. In this process nitrogen is bubbled into the liquid (water or electrolyte) while being subjected to an atmospheric pressure plasma discharge, enabling the transport of the activated species within the liquid. These species (particularly nitrates and nitrites) can be then efficiently converted into ammonia by using a designed electrocatalyst.

In addition to the reduction of nitrogen, it should be noted that the process can be advantageously used in electrocatalytic carbon dioxide reduction reaction, which requires transformation of stable carbon dioxide molecules into comparatively more energetic and reactive states, which this invention can provide thus delivering enhanced performance as well as controllable selectivity.

A particular advantage of the present invention may be found where in reactions where the gas phase activation is the rate determining step.

As aforementioned, two of the most significant inhibitors to electrocatalytic NRR (eNRR) are the high stability and low solubility of the N₂ molecule in liquids. The present invention seeks to overcome these limitations by converting N₂ into a more reactive and soluble form.

Nitrate and nitrates are highly soluble and much more easily reduced to ammonia than N₂. While this approach may seem promising, it needs to be kept in mind that the industrial process for producing nitrates and nitrates are is from ammonia via the Ostwald process, thus their direct use as precursors for ammonia production is highly circuitous and impractical. Additionally, nitrates/nitrites have limited stability in water hence direct production and on-spot utilisation is desirable. Consequently, the production of NOₓ via a plasma-driven process for the direct consumption to produce ammonia would be a desirable industrial process, if practicable.

The first step in the process of the present invention is the plasma-activation of air, at the water/electrolyte interface, to produce NOₓ (i.e. a mixture of NO₂_{^{−}} and NO₃_{^{−}} species). Plasma is essentially an ionized gas composed of a range of species (including electrons, ions, radicals, molecular fragments) at various energy levels.

Plasma can be categorized into thermal and non-thermal plasmas (NTP). Thermal plasmas exhibit equilibrium between electrons and bulk gas temperatures (typically higher than 5×10³ K). Meanwhile, in NTP such equilibrium is not established thus the temperature of the electrons can be several orders of magnitude higher than ambient. NTP is less energy intensive than thermal plasmas, and still possess electrons with high translational energies required to overcome the stability of the N₂ molecule via electronic structure transitions, which makes NTP a suitable choice for aforementioned process.

Nitrogen activation, and oxidation, is difficult due to the thermodynamic and kinetic stability of nitrogen in the energy required to break the N₂ triple bond. Plasma is able to provide sufficient energy to activate N₂. This reaction occurs commonly in nature as a consequence of lightning to produce NO.

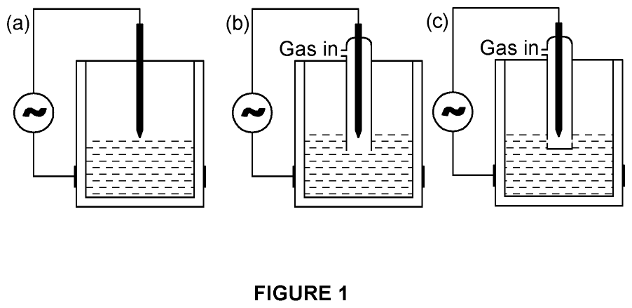

Three key approaches have been studied in order to drive the plasma-driven generation of NOₓ species. FIG. 1 depicts various configurations of plasma discharge (a) pin to liquid discharge, (b) pin-in-nozzle discharge and (c) bubble discharge. The purpose of these systems is to generate plasma at the liquid/gas interface, producing NOₓ species which can then be dissolved into the water/electrolyte.

There are a range of variables which in can be used to control the (i) amount of species, (ii) the overall energy efficiencies of the system (NOₓ produced/power input) and (iii) ratio of nitrate to nitrite. These variables include plasma input voltage (amplitude, pulse width and repetition frequency), time, gas flow rate, and liquid flow rate.

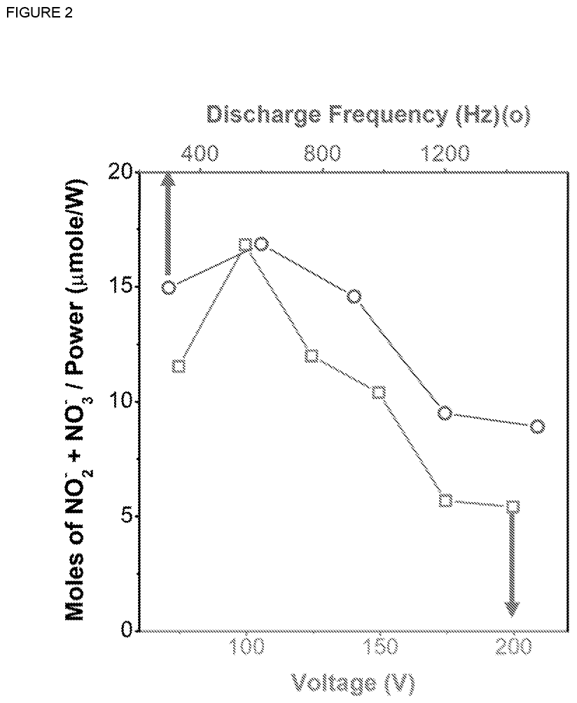

The impact of these parameters of the performance of the different plasma systems are interrelated. For example, the impact of changing voltage and pulse/discharge frequency on the pin-in-nozzle design on the total quantity of NO produced is relatively minor, as shown in FIG. 2. This is attributed to the mass-transfer of the activated species in the solution being the limiting factor in NOₓ production, as opposed to the plasma itself. On the other hand, when those mass transfer impacts are overcome, by implementing a bubbler-system of the present invention the impacts of varying parameters become much more significant.

Table 1 demonstrates a sample results comparison between the differing designs for plasma NOₓ generation, specifically comparing the pin-in-nozzle and column bubbler. It is clear that the production rate is significantly higher in the case of the column bubbler, however, in this case the ratio of nitrates/nitrates is notably different.

TABLE 1
NOₓ generation results for the pin-to-liquid in nozzle enclosure and
pin-to-liquid in column bubbler (Refer to FIG. 1, b and c, respectively)

| | | Pin-to-liquid |
|---|---|---|
| | Pin-to-liquid in nozzle | in column |
| | enclosure (b) | bubbler (c) |
| Power (W) | 10 | 33 |
| Time of operation (min) | 15 | 5 |
| Volume of electrolyte (L) | 0.1 | 0.25 |
| Type of electrolyte | Water + 0.1M HCL | Water |
| Air flow rate (L/min) | 0.03 | 1 |
| NO₂⁻ concentration (mM) | 1.2 | 0.34 |
| NO₃⁻ concentration (mM) | 0.5 | 1.61 |
| NO₃⁻ / NO₂⁻ ratio | 0.41 | 5.3 |
| Total NOₓ (mM) | 1.7 | 1.91 |
| Production rate | 0.068 | 0.174 |
| (moles/kWh) | | |

Ultimately, the design of the plasma-system, along with the input voltage, frequency, time, gas type and flow rate, humidity and temperature liquid type (i.e. electrolyte/water) and flow rate all have a significant impact on the quantity of NOₓ, the energy efficiencies of the species produced (NOₓ produced/power input) as well as the ratio of nitrate to nitrite. Given the teachings in the present specification, it would be expected that variations in design to optimise NOx production would be within the capacity of a person of ordinary skill in the art.

Attention was then focussed enhancing energy efficiency with respect to NOₓ formation. An AC sinusoidal waveform with periodic gaps between discharges was employed as compared to DC plasmas, AC waveform is considered more efficient, inexpensive, and reliable for longer operation. This is because of higher excited state active species at similar powers (in some cases 5 times higher) due to less energy dissipation into heat at the electrodes in aqueous environments. Compared to thermal plasmas, where gas is heated to temperatures typically in order of 20,000 K, the present invention utilized non-thermal (cold) plasma which is generated at ambient temperatures and pressures but still exhibit elevated electron temperatures.

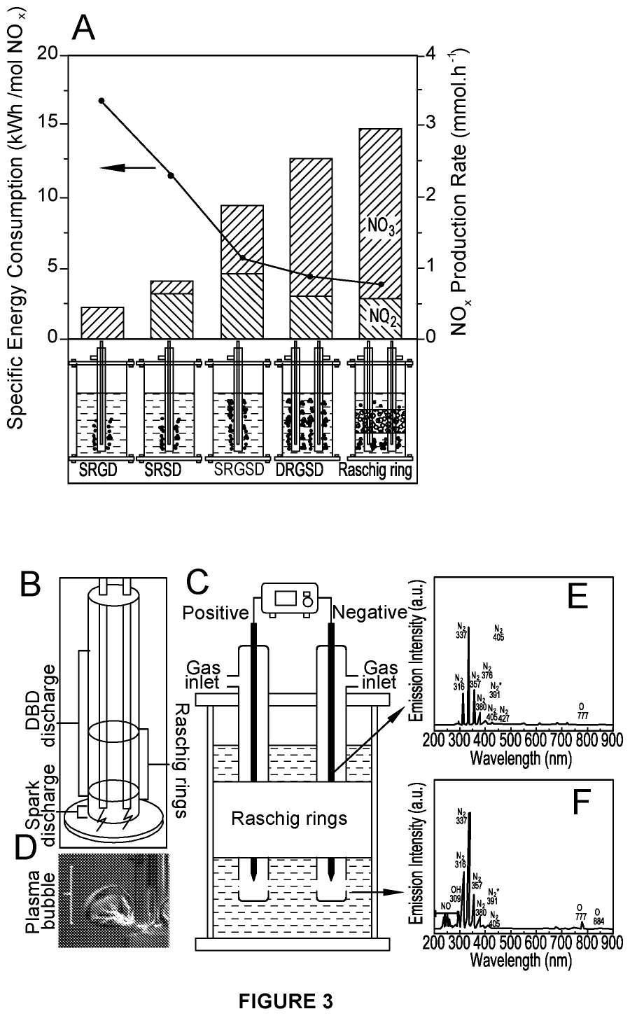

The use of underwater plasma bubbles intensifies gas-to-liquid mass transfer, which is assisted by interfacial areas, residence time and internal pressures. Thus, the present inventors developed a non-thermal AC plasma, exploiting a bubble column with varying discharge regimes (including spark and glow discharges). Five different design configurations (FIG. 3A) were tested, with underwater plasma bubbles as the dominating aspect of the design.

It is clearly shown in FIG. 3A, that the combination of two reactors, exploiting both spark and glow discharges, coupled with Raschig rings resulted in a significant increase in NOₓ energy efficiency, attaining a NOₓ generation energy efficiency of 263 mmol kWh⁻¹ which is three times better than the state of the art.

The key features of the reactor design (FIG. 3B-D), leading to high energy efficiencies are characterised by the combination of (a) multiple discharge schemes (glow and spark discharges) (b) dual reactor configuration within one AC circuit; and (c) bubble dynamics control (Raschig rings). As shown in FIG. 3E&F, the OES data indicates that the excited species generated in the glow discharge differ drastically from those in the spark discharge. Within a glow discharge system, NO₃ is the dominant species (SRGD) produced, whilst with the spark discharge, NO₂_{^{−}} is favoured (SRSD). Combining the two forms of discharges within a single unit made efficient utilization of the applied power minimizing energy losses. Further, the implementation of dual reactors (one being the high voltage electrode, the other being the ground), leads to a reduction in energy losses resulting from having the ground reactor within the solution.

The incorporation of Raschig rings further increased the energy efficiency. This enhancement can be attributed changes in mass transfer and residence times, allowing for an intensification of mass transfer from the gas phase NOₓ species into solution. Ultimately, these key design approaches resulted in an energy efficient, scalable approach to aqueous NOₓ production.

Once the NOₓ (nitrate/nitrite mixture) is produced from the plasma, it needs to be reduced and the method chosen in the present case is electrocatalytic reduction. In order to better understand the system, and, the ability of the NOₓ intermediaries to be electrocatalytically reduced to ammonia, the present inventors initially focused on H-cell experiments. In these experiments, the electrocatalytic conversion of NOₓ to ammonia was performed using an integrated system that incorporates a custom-design plasma-bubbler to the electrochemical H-cell, as well as with NOₓ salts, to understand the electrocatalytic conversion pathways.

The conversion of NOₓ species can produce ammonia at higher rates and faradaic efficiencies than N₂ directly. In acidic media, the reaction proceeds as shown in Equations 1-3 below. The reaction competes with the hydrogen evolution reaction (HER), Equation 4. Whilst HER occurs at more negative potential than the nitrate/nitrite reduction, slow kinetics for nitrate/nitrite reduction may lead to HER occurrence and it has been found that unwanted HER may be addressed by electocatalytic optimization.

| | | | |
|---|---|---|---|
| NO₃⁻ + 2H⁺ + 2e⁻ → NO₂⁻ + H₂O | E⁰ = 0.83 V | E⁰ = 1.02 V | (1) |
| | vs NHE | vs RHE | |
| NO₃⁻ + 10H⁺ + 8e⁻ → NH₄⁺ + 3H₂O | E⁰ = 0.87 V | E⁰ = 0.93 V | (2) |
| | vs NHE | vs RHE | |
| NO₂⁻ + 8H⁺ + 6e⁻ → NH₄⁺ + 2H₂O | | E⁰ = 0.86 V | (3) |
| | | vs RHE | |
| 2H⁺ + 2e⁻ → H₂ | E⁰ = 0 V | E⁰ = 0 V | (4) |
| | vs NHE | vs RHE | |

Thus, the different pathways for the conversion of NOₓ, the competing HER as well as different reactants (NO₃_{^{−}} and NO₂_{^{−}}) mean that catalyst design for ammonia synthesis requires careful consideration.

The present invention has established that a range of transition metals can be used to facilitate the electrocatalytic conversion of nitrogen to ammonia. Of these copper and nickel were the most preferred. The description of the electrocatalyst will be provided with reference to copper but it will be appreciated that it can apply to other transition metals.

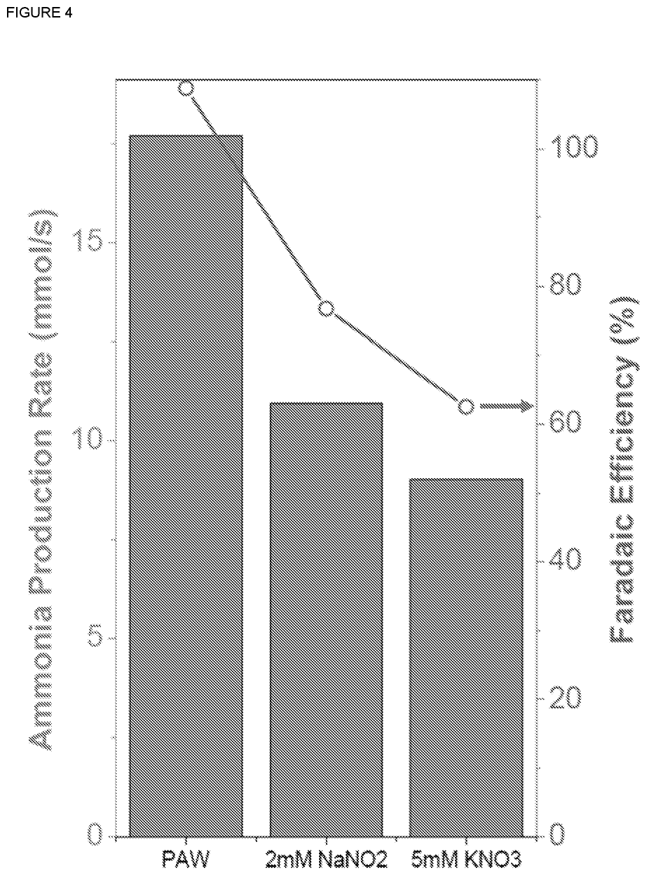

In the present invention, as found in FIG. 4, Cu foil is capable of effectively converting NOₓ to ammonia at high Faradaic efficiencies with high production rates. By studying a number of forms of copper, such as Cu foam, Cu nanostructures and single atom Cu catalysts, a clear correlation between Cu surface chemistry and surface area can be established.

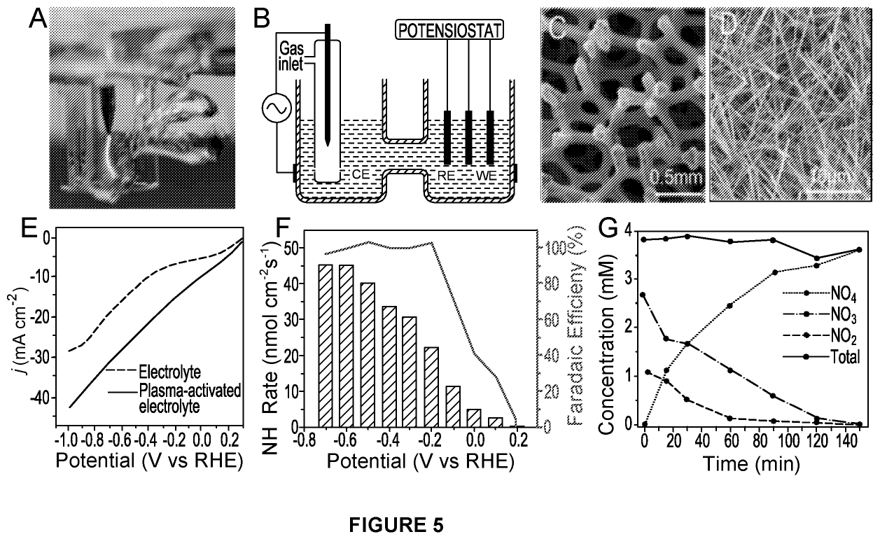

A range of Cu-based catalysts was prepared and evaluated for their performance for the electrocatalytic conversion of NOₓ to ammonia (Cu foil, foam and nanowires (NWs) grown on foam). Representative scanning electron microscopy (SEM) images of the Cu NWs (FIG. 5 C&D) supports the existence of a nanoporous morphology of uniformly dispersed thin nanowires on the copper foam. It is clear from these images that the Cu NWs seed out from the metallic Cu skeleton of the porous background foam during electrode preparation.

It was found that the Cu NWs sample was able to attain the highest current density (j) for the reduction of plasma-activated electrolyte whilst achieving an ammonia production rate of 45 nmol·s⁻¹·cm⁻² and Faradaic efficiency (FE) of ˜100%.

Comparatively, the Cu foil and foam facilitated somewhat lower FEs of ˜80 and 71%, respectively, with ammonia yields of 6.0 nmol·s⁻¹·cm⁻² and 8.9 nmol·s⁻¹·cm⁻² (at −0.5 V). This variation in catalytic activity can be ascribed to the variation in electrochemical active surface area (ECSA) between the electrodes.

The ECSA for the Cu NWs catalyst was significantly larger for the foil and foam samples, indicating an increase in active sites for the NWs sample, ultimately improving the overall yield of ammonia. The high FE is due to the presence of Cu¹⁺/Cu⁰ is well-known for the suppression of the competing hydrogen evolution reaction HER, which is the breakdown of water into oxygen and hydrogen.

To understand the origin of the NH₃, in particular whether it arises from NOₓ or dissolved N₂ reduction, control experiments were performed showing that the electrolyte alone (with no plasma activation) resulted in no NH₃ production. Furthermore, the polarisation curves (FIG. 5E) revealed that the Cu NWs electrode facilitated a very high current density (j), attaining a j of −45 mA cm⁻² at −1 V (compared to 28 mA cm⁻² for the blank electrolyte). This indicates that the current obtained arises from eNRR and not from competing HER.

FIG. 5F displays the dependence of ammonia production rate and FE for NOₓ reduction on applied potential (each electrolysis duration was 0.25 h). As the potential was changed from 0.2 V to −0.6 V, the ammonia production rate increased along with the FE (from 5% at 0.2 V to ˜100%). The lower FE (<100%) between 0.2 V to —0.2 V can be ascribed to some charge loss arising from the conversion to NO₃_{^{−}} to NO₂_{^{−}} species. During NO₃_{^{−}} reduction, adsorbed *NO₂ was identified as a key intermediary.

It was observed a portion of the *NO₂ desorbed into the solution as NO₂_{^{−}} at lower potentials, hence the observed lower FE at lower potentials (between 0.2 to −0.2 V). At higher potentials, however, the conversion rate of both nitrate and nitrite to ammonia is very high, which compensates for this side reaction.

To further understand the reaction pathway, and the consumption of both nitrate and nitrite as a function of electrolysis duration, a batch experiment was undertaken with a successive sampling of nitrite, nitrate and ammonia (FIG. 5G). With an extended reaction time of 2.5 h, both NO₃_{^{−}} and NO₂_{^{−}} species were exhausted entirely (from 2.7 mM and 1 mM, respectively). On the other hand, ammonia concentration increases from 0 mM to 3.5 mM over the same period. The total concentration of N-species remained constant during electrolysis. Moreover, the chronoamperometric i-t curve displays a consistently declining j which indicates the consumption of reactants during eNRR. Throughout the prolong experiment the FE remained at ˜100% for the first 1 h then slowly declined as the reactive NOₓ are consumed and converted completely into ammonia. Importantly, post-reaction assessment of the Cu NWs cathode through X-ray photoelectron spectroscopy (XPS) revealed no noticeable chemical changes of the electrode. Moreover, the N1s spectrum did not indicate any nitrogen bonded to the surface of the cathode, indicating the non-poisoning interaction of the NOₓ reactive species with the catalyst.

It is highly desirable to integrate the plasma-driven production of NOₓ with the electrocatalytic system for the production of ammonia. The stability of the produced nitrate/nitrites is low and thus the direct conversion of the activated species to ammonia is highly desirable.

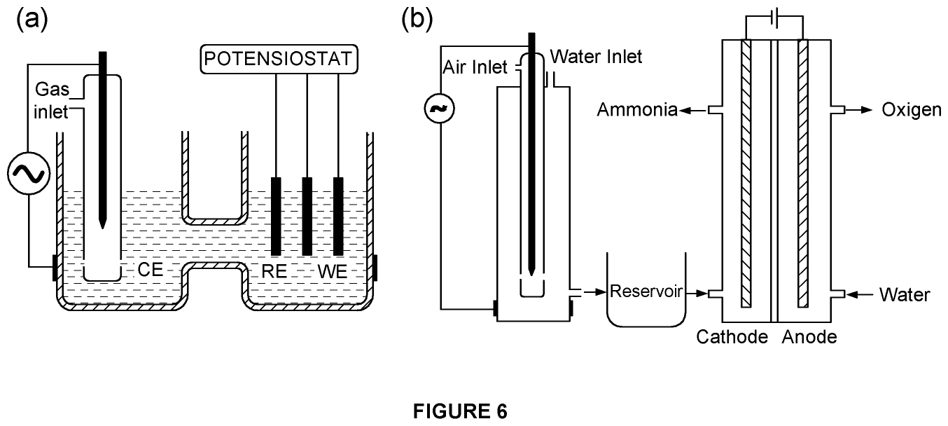

Two possible approaches to integrating the plasma/electrocatalytic systems as shown in FIG. 6. FIG. 6a displays the incorporation of the plasma-bubbler to a batch-type H-cell system, used for lab scale validation. FIG. 6b shows flow through system with the plasma-bubbler having a liquid outlet leading to a flow through electrolyser to convert the NOₓ to ammonia. It should be noted that the optimization of the NOₓ production, relative to the NOₓ consumption in the electrolyser is required to maximize production and overall energy efficiency. A further benefit to note is the direct production of fertilizer (i.e. ammonium nitrate) from the system. The integration is rather intricate because of simultaneous electrodynamic of both AC-driven plasma source and DC-driven electrocatalysis source. This requires appropriately balancing the current flow within the two aforementioned electric circuits as well as mass and energy balances for achieving optimal throughputs. When the appropriate production/consumption balances are put in place, it is possible to directly produce ammonium nitrate from the conversion.

Alternative embodiments are envisaged in which a reservoir is provided intermediate the NOₓ generation vessel and the NOₓ reduction vessel. A reservoir can provide benefits in terms of feeding the NOₓ for reduction at a predetermined rate, which can avoid build-up of NOₓ or NOₓ starvation at the site of electrocatalytic reduction.

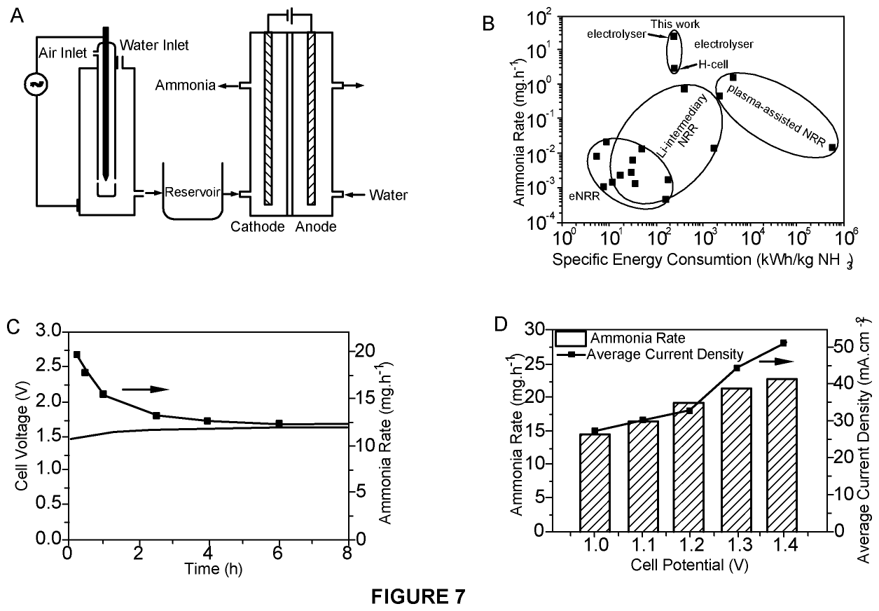

The present system was tested and it was established that an increase in cell voltage from 1 V to 1.4 V resulted in an increase in j from 27 mA cm⁻² to 52 mA cm⁻² and ammonia rate from 15 mg h⁻¹ to up to 24 mg h⁻¹. Furthermore, the stability of the flow system at a current density of 30 mA cm⁻² was investigated, where plasma-activated electrolyte was fed continuously while ammonia was collected from the outlet. The hybrid system maintained a stable applied cell voltage of 1.5±0.04 V and an average Faradaic efficiency of —58% for 8 h continuously (FIG. 7C).

The electrochemical conversion of the resultant NOₓ intermediaries, using a scalable electrolyser, resulted in current densities of over 50 mA/cm², Faradaic efficiencies of ˜60%, an ammonia production rate of 23.2 mg/h (42.1 nmol/scm²) at a very low cell voltage of 1.4 V.

FIG. 7B compares the overall production rate of ammonia with recently reported state-of-art results for eNRR, Li-intermediary NRR, and plasma-assisted NRR demonstrated at ambient conditions. The NOₓ intermediary approach developed in this study is shown to facilitate the highest potential to yield high rates of ammonia while maintaining high energy efficiency. The ammonia yield rate is between one to three orders of magnitude higher than every other electrochemical method (at similar reaction geometric areas). When scaled up using an electrolyser, the rate increased by another order of magnitude. At the same time this hybrid system is characterized by much reduced power consumption (total of 253 kWh/kg NH₃) compared to plasma-assisted ammonia production technologies. This is between one to three orders of magnitude less energy-intensive than plasma assisted electrochemical conversion of nitrogen to ammonia and gas-phase Dielectric Barrier Discharge (DBD) synthesis method. The energy consumption is also better than the studies that showed relatively high yield ammonia production via Li-intermediary approaches. In the case of eNRR, no practical method has been demonstrated until now to show a considerable production rate and FE, making those systems unfavourable for scaling.

### EXAMPLES

### GENERAL EXPERIMENTAL

### Materials

All reagents and solvents were purchased from Sigma-Aldrich or from Chem-Supply Pty Ltd. Cu foam was purchased from Xiamen TMAX Machine Limited. Oakton pH/Ion 700 Ion 700 Benchtop Meter and Cole-Parmer Combination Ion Selective Electrodes (nitrate) were purchased from John Morris Group. Milli-Q water with a resistivity of 18.2 MΩ·cm was obtained from an inline Millipore RiOs/Origin H₂O purification system, was used throughout the experiments for sample preparation and reaction.

### Copper Nanowires (Cu NWs) Fabrication

The commercial Cu foam and foil was cut into desired sizes and ultrasonically cleaned with acetone, ethanol, and finally Milli-Q water for 15 min intervals, and then washed with dilute H₂SO₄ solution to remove any surface impurities and oxide layers. Cu(OH)₂ nanowires were first synthesized on Cu foam by immersion into a solution containing 0.133M (NH₄)₂S₂O₈ (ammonium persulfate) and 2.667m NaOH for 0.5 h at room temperature. Subsequently, the Cu foam was removed out from solution, rinsed with Milli-Q water and absolute ethanol, and air-dried. CuO NWs were then fabricated by annealing the prepared Cu(OH)₂ NW arrays at 180° C. for 1 h in air. The resulting CuO NW sample was electrochemically reduced to Cu/Cu₂O NW arrays in 0.5M Na₂SO₄ under −1V vs RHE.

### Electrochemical Evaluation

All electrochemical evaluations were conducted using Autolab Potentiostat (Autolab M204) in a custom-designed H-type electrochemical cell and electrolyser. The cathodic chamber was separated from the anodic chamber by Nafion© 117 membrane. For the H-type cell, a three-electrode set-up using the Cu catalyst (foil, foam and Cu NWs) as the working electrode (WE), platinum wire as the counter electrode (CE) and Ag/AgCl (sat. KCl) reference electrode (RE) was used. 10 mM H₂SO₄ was used as the background electrolyte in this study, and the optimization of acid concentration was performed. Typically, for the H-type cell studies, 50 mL of electrolyte was used in the cathodic chamber to allow for electrolyte sampling. The electrode size for the H-cell was 1 cm⁻² and Cu foil was used for optimization studies. The reaction was facilitated with magnetic stirrer at the speed of 650 rpm. All potentials for H-type cell were described versus the reversible hydrogen electrode (RHE) via the following equation:

E_{RHE}=E_{(Ag/AgCl)}+0.197+0.059×pH(pH=1.68 in this study)

To further translate this concept for large scale application, the plasma-activated water (PAW) from the scaled-up reactor (vide infra) was fed into a high throughput electrolyser to understand the potential for ammonia production rate and yield. A membrane electrode assembly (MEA) was prepared by sandwiching the Cu NWs cathode (electrode size 9 cm²) and Ru/TiO₂ anode between a commercial Nafion membrane. The MEA was loaded within the electrolyser with PAW being used as the catholyte and 0.1 M H₂SO₄ as the anolyte (using a peristaltic pump with a flow rate of 1.5 mL/min). For the electrolyser optimization, 250 mL of the PAW was circulated in the cathodic chamber. For the stability test, a continuous flow was used for 8 h with 30 mA·cm⁻² being applied.

### Ammonia (NH₃) Detection by the Indophenol Blue Method

From the cathodic chamber electrolyte solution, 0.5 mL of electrolyte was taken and transferred into a 2 mL sample tube. Into the tube, 0.4 mL of 1 M NaOH solution (with 5 wt. % salicylic acid and 5 wt. % sodium citrate), 0.1 mL of 0.05 M NaClO and 30 μL of 1 wt. % C₅FeN₆Na₂O (sodium nitroferricyanide) in water was added. The mixture was then incubated in the dark at room temperature for 2 h prior to UV-Vis testing. The concentration of ammonia was determined via a calibration curve. The calibration curve was prepared using a set of standard solutions with a known amount of (NH₄)₂SO₄ (concentrations were based on NH₄⁺) in 10 mM H₂SO₄. Into these solutions, the above-mentioned indophenol blue reagents were added, and the indophenol blue absorbance at 655 nm was determined after 2 h. The limit of detection (LOD) of UV-Vis used in this study refers to the absorbance at 655 nm obtained from blank 10 mM H₂SO₄ for the lower limit and from 200 μM NH₄⁺ for the upper limit.

### Nitrite (NO₂⁻) Detection by Griess Reagent

50 μL of the sample was taken and transferred into a cuvette and combined with 50 μL of Griess Reagent and 0.9 mL of Milli-Q water. The resulting sample was mixed thoroughly. The mixture was incubated at room temperature in the dark for 0.5 h prior to UV-Vis testing. Solutions of NaNO₂ with known concentrations (in 10 mM H₂SO₄) were used as calibration standards, with the absorbance at 525 nm used to plot the calibration curves. Upper LOD of UV-Vis used in this study refers to the absorbance at 525 nm obtained from 200 μM NaNO₂. A dilution factor was applied to measure nitrite concentration in plasma-activated water (PAW).

### Nitrate (NO₃⁻) Detection by Ion-Selective Electrode

An ion-selective electrode (ISE), also known as a specific ion electrode (SIE), is a transducer (or sensor) that converts the activity of a specific ion dissolved in a solution into an electrical potential. The voltage is theoretically dependent on the logarithm of the ionic activity, according to the Nernst equation. Cole Palmer Nitrate selective probe has a concentration range of 7 μM to 1M (0.5 to 62,000 ppm). The ionic strength of ion solutions varies with the concentration of the ion to be measured. To maintain a constant ionic strength, an Ionic Strength Adjuster (ISA) is added. This ensures the total ionic strength is independent of the analyte concentration. In this study, 2M ammonium sulfate (NH₄)₂SO₄ was added, as the ISA, at 400 μL to each 20 mL of standard or sample to adjust the ionic strength to about 0.12 M.

### H₂ Detection by Gas Chromatograph (GC)

H₂ detection was tested by GC (Shimidzu, Model 2010 Plus) equipped with both thermal conductivity detector (TCD) and flame ionization detector (FID) detectors.

### Physical Characterization

X-ray photoelectron spectroscopy (XPS) was performed on a Thermo Scientific K-Alpha X-ray spectrometer. The morphology and structure of Cu NWs were imaged by scanning electron microscopy (SEM) using a JEOL JSM-IT-500 HR. UV-Vis absorption spectra were recorded on a Shimadzu UV-3600 UV-VIS-NIR spectrophotometer.

### Faradaic Efficiency and Production Rate Calculations

The two crucial descriptors, revealing ammonia synthesis performance, are the Faradaic efficiency and the ammonia production rate. The Faradaic efficiency indicates the selectivity of the electrocatalysis for ammonia synthesis, which refers to the ratio of the electrical energy consumed for the synthesis of ammonia to the overall energy through the electrochemical system. The Faradaic efficiency (η) of ammonia synthesis was determined by Eq. (51), where n is the figure of the desired electrons for synthesizing one ammonia molecule (n=6 when ammonia is from nitrite and n=8 when ammonia is from nitrate), F is the Faraday constant (F=96485.33), C is the detected ammonia molar concentration, V is the electrolyte volume, and Q is the overall electrical energy travelled over the electrodes. To calculate the number of the exchanged electron, both nitrite and nitrate concentrations were measured before and after each reaction; the average found to be 7.6. For the reactions that both nitrite and nitrate are fully exhausted, n was calculated based on their initial ratio.

$$η = \frac{n \cdot F \cdot C \cdot V}{Q} \qquad ( S1 )$$

Ammonia production rate (R) is the ammonia production over unit time and over unit electrode surface area. It can be determined by Eq. (S2), where C is the detected ammonia molar concentration, V is the electrolyte volume, t is the reaction time, and S is the catalytically active surface area of the electrode.

$$R = \frac{C \cdot V}{t \cdot S} \qquad ( S2 )$$

Plasma activation of water in the H-cell.

Ground-state nitrogen molecules exhibit high ionization potential making it intrinsically unreactive from a thermodynamic standpoint. Still, plasma activation provides avenues for the conversion of highly stable nitrogen molecules into easier to breakdown intermediaries (NOₓ). Plasma can be categorized into thermal and non-thermal plasmas (NTP). Thermal plasmas exhibit equilibrium between electrons and bulk gas temperatures (typically higher than 5×10³ K). Meanwhile, in NTP such equilibrium is not established; thus the temperature of the electrons can be several orders of magnitude higher than ambient. NTP is less energy-intensive than thermal plasmas, and still possess electrons with high translational energies required to overcome the stability of the N₂ molecule via electronic structure transitions, which makes NTP a suitable choice for the aforementioned process.

The design of the plasma-system, along with the input voltage, frequency, time, gas type and flow rate, liquid type (i.e. electrolyte/water) and flow rate all have a significant impact on the quantity of NOₓ, the energy efficiencies of the species produced (NOₓ produced/power input) as well as the ratio of nitrate to nitrite.

For the batch electrochemical tests, custom plasma bubbler was used in an H-cell and connected to the plasma generator (‘Leap 100’ from PlasmaLeap Technologies). The optimized plasma generator parameters were using a voltage of 100V, duty of 83 μs, discharge frequency of 600 Hz and resonance frequency of 60 kHz. Dry air (Coregas, dry air) was introduced from the top of the custom plasma bubbler at 20 mL/min to generate PAW. The plasma activation was performed for 0.5 h to achieve NOₓ concentration of ˜4 mM in 100 mL water.

### Plasma Discharge Design

### Plasma Reactors, Discharge Schemes and Configurations

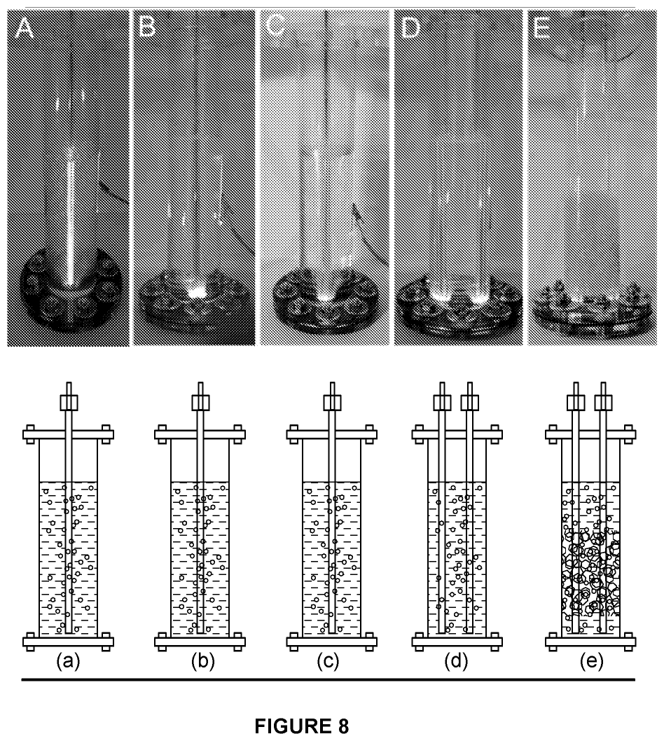

Five reactor design configurations using underwater plasma bubbles were tested. Photographs of plasma bubble column reactors design configurations are shown in FIG. 8, were: (a) single reactor glow discharge (SRGD); (b) single reactor spark discharge (SRSD); (c) single reactor glow and spark discharge (SRGSD); (d) double reactor glow and spark discharge (DRGSD); and (e) DRGSD with

Raschig rings. Plasma bubble column reactors were capable of dual-discharge mode operation. i.e. glow and spark discharge. To achieve the former, the high voltage electrode was sheathed with borosilicate. The latter incorporated a sharpened high voltage electrode with a 1 cm protrusion which induced a spark extending longitudinally towards the bubbles. Meanwhile, combinative discharge reactors coupled both these concepts in a single unit. In configurations involving single reactor, water was used as ground, meanwhile double-reactor configurations utilized secondary plasma reactor as ground. Plasma reactors were fabricated using a quartz tube with one end sealed and 12 laser-drilled holes with a diameter of 200 μm located radially 5 mm above the sealed base. Stainless steel rod was used as high voltage electrode inserted concentrically into the quartz tube. A tee fitting was connected to the quartz tube to position the electrodes. Instrument grade air was injected as the feed gas at a flow rate of 1 L/min in each reactor via a mass flow controller. Reactors were powered by plasma generator (‘Leap100’, PlasmaLeap Technologies) capable of yielding voltage output of 0-80 kV (peak-to-peak), discharge power of up to 700 W, and a discharge frequency range of 100 Hz-3000 Hz. For all experiments, power was provided in form of batches of sinusoidal pulses with a lag time between each batch. Resonance frequency of pulse was set at 60 kHz while discharge frequency of each batch of pulses was 300 Hz (duty cycle of 103 μs).

Electrical and optical measurements. A digital oscilloscope (DS6104, Rigol) was employed to record both the sinusoidal voltage and current waveform via a high voltage probe (PVM-6, North Star) and a current probe (4100, Pearson), respectively. The time-averaged discharge power (P) was calculated from the measured discharge voltage and current with the following formula:

P=f∫ₜ_{₀}^{t}^{₀}^{+T}u(t)i(t)dt

The electrical parameters across various reactor configurations are presented in Table 2. Optical Emission Spectra (OES) were recorded using a spectrometer (SR-500i-A-R, Andor Shamrock), with a grating groove of 300 lines mm⁻¹ and exposure time of 20 ms.

TABLE 2
Operational conditions of plasma reactors and production rates of species

| | Voltage | Current | | Operation | Mean | Mean | | |
|---|---|---|---|---|---|---|---|---|
| | amplitude | amplitude | Power | time | NO₃ | NO₂ | Conductivity | |
| | (kV) | (A) | (W) | (min) | (ppm) | (ppm) | (μS/cm) | pH |
| SRGD | 12.5 | 0.38 | 7.38 | 10 | 4.6 | 0 | 37.70 | 4.02 |
| SRSD | 9.8 | 0.5 | 9.22 | 10 | 1.8 | 4.8 | 38.37 | 4.00 |
| SRGSD | 8.7 | 1.56 | 10.67 | 10 | 9.9 | 7 | 107.47 | 3.54 |
| DRGSD | 8.5 | 1.68 | 11.27 | 10 | 20.2 | 4.5 | 170.20 | 3.37 |
| DRGSD + | 8.4 | 1.68 | 11.27* | 10 | 24.9 | 4.3 | 209.90 | 3.29 |
| Raschig | | | | | | | | |
| rings | | | | | | | | |

Using an AC system was desirable as polarity inversion in AC systems prompts current passing through zero at half cycle which enhances the lifetime of electrodes.

Cold plasma is particularly useful to selectively transfer incident electric power to the electrons rather than volumetric heating of the entire gas as it is an energy-efficient route to formation of active species via collisions.

Without wishing to be bound by theory, it is believed that higher production of reactive radicals in the aqueous phase takes place because of mechanical agitation and local heating caused by bursting of bubbles.

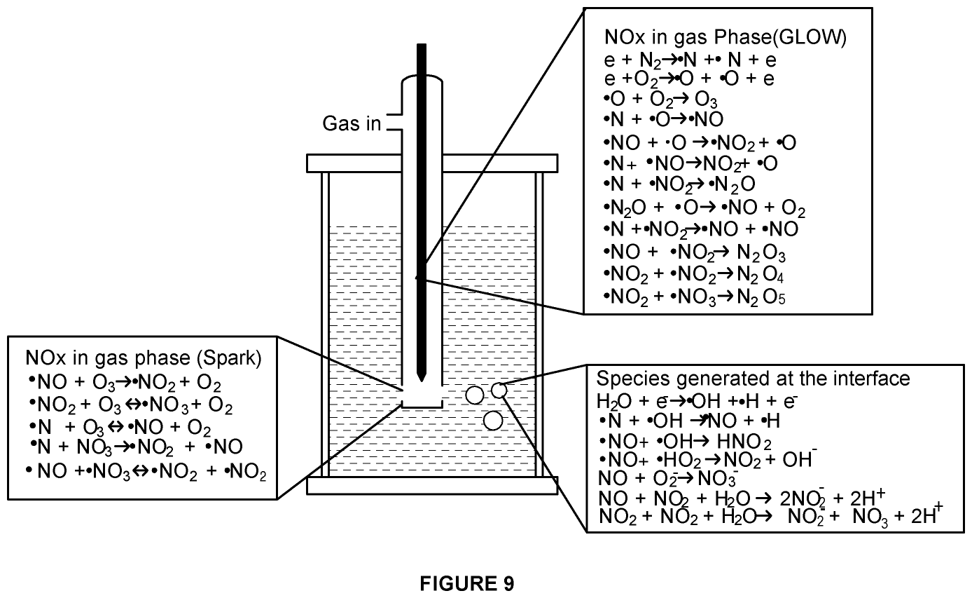

FIG. 9 shows the plasma processes arising from Spark and Glow plasma ionization of N₂, and the species generated at the interface.

Single reactor glow discharge (SRGD) was operated at glow-only discharge scheme by applying 7.38 W power and using a dielectric barrier around the high voltage electrode. In principle, the use of dielectric barrier limits the flow of charge enabling higher voltages at the same power. In such discharge scheme, production of NO₃ predominated over NO₂, which corroborates with literature. Meanwhile, spark-only discharge scheme was dominated by NO₂ over NO₃, and higher current to voltage ratios than the glow-only scheme and comparatively higher power (9.22 W). High intensity electric fields in glow discharge scheme favour ozone production, which maintain oxidation environment the entire volume in the tube facilitating conversion of NO₂ to NO₃. However, spark streamers are confined in the concentrated volume prompting formation of high energy species and back-reactions of NO₃ to NO₂.

Thus, the underwater plasma bubbler reactors of the present invention , combine both glow and spark discharges to generate the NOₓ intermediaries at the unprecedented energy efficiency of 263 mmol/kWh.

It should be noted that the above sections outline the use of air as the inlet gas, however, to date a range of different gases have been examined. It is possible to use any gas and tune the plasma/inlet parameters to obtain the desired product. In the present specification, the results presented are predominantly for the use of air, however mixtures of N₂/O₂, along with H₂O also show promise. It should be further noted that the system has also been adapted for use for CO₂ conversion showing favourable results.

### Catalyst

### Incorporation of Catalyst into the Plasma System

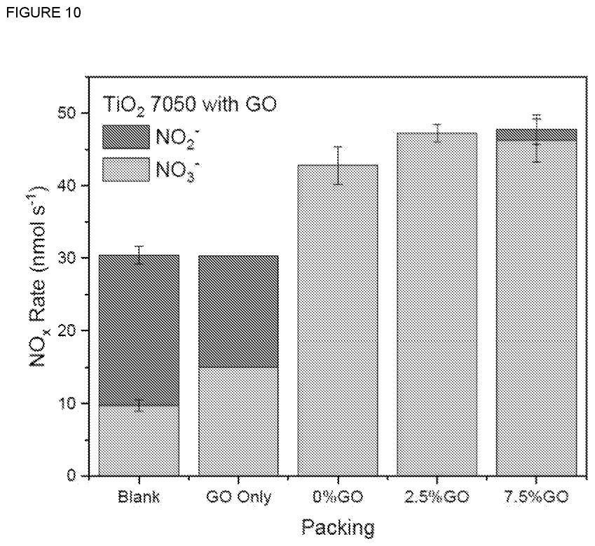

The incorporation of a catalyst into the glow and/or spark discharge region for the plasma-driven NOx generation. FIG. 10 below displays the impact of incorporating a metal oxide (TiO₂) with an appropriate binder (Graphene Oxide, GO) for the plasma-driven NOx synthesis. It can be seen that total NOx production rate was increased by ˜50% and the concentration of GO has negligible effect on the performance. GO works as the binder for shaping the metal oxide catalyst as the packing for plasma reactor system.

As mentioned above, transition metal catalysts, an specifically copper, nickel, tin, iron, bismuth, cobalt, titanium and oxides thereof are particularly useful in the present invention.

Any suitable catalyst binder, such as silica, alumina, clays, polymers or carbon based supports can be used.

### Physical Characterization of Catalytic Sites

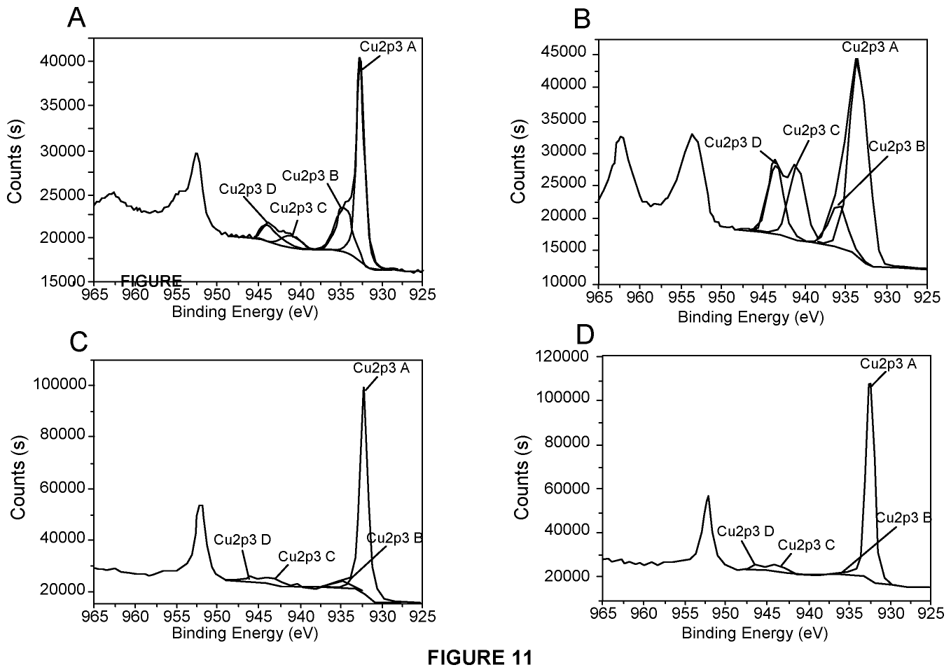

To probe the active sites responsible for eNRR, XPS analysis was carried out on the post-reaction Cu NW electrode to investigate any variation in the surface chemical state of the electrode owing from the negative bias applied herein. From FIG. 11 it can be seen that the Cu²⁺ species are reduced to Cu¹⁺/Cu⁰ (indicated by the peak shift to 932.6 eV). On the basis of these results and eNRR data, and without wishing to be bound by theory, it is believed that these interfaces are playing the role of active sites for eNRR reaction. It is understood that the formation of Cu¹⁺/Cu⁰ interfaces in Cu-based catalysts leads to a suppression of the competing hydrogen evolution reaction (HER) during eNRR. Further, these interfaces promote eNRR by reducing the free energy barrier for ammonia formation through nitrate and nitrite ions. The XPS results reveal that the surface of the nanowires comprises of mostly of CuO species, evident from the high-resolution Cu 2p_{3/2} spectra which show a large peak at binding energy 933.7 eV which is ascribed to Cu²⁺.

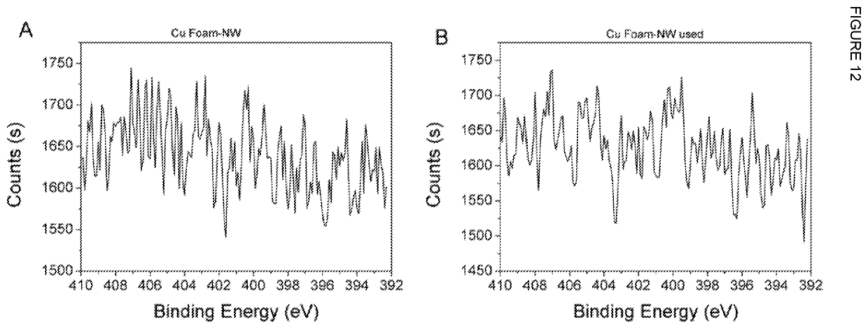

FIG. 12, showing the N1s spectra, clearly indicates that there was no nitrogen attachment to the surface of post-reaction Cu NW electrode. This indicates that the Cu NWs catalyst did not suffer from poisoning by the reactant NOₓ species and supports the stability of the catalyst.

### NOX Analysis

### Calibration Plot and Background Determination

A number of calibration and control experiments were conducted using UV-visible spectroscopy to investigate the background of NH₃ and NOₓ in the used electrolytes, PAW, solutions and electrodes,

It was observed that a in some bubbler configurations a barely detectable amount of ammonia is generated at the rate of 0.21 nmol s⁻¹ during the plasma activation, however, with the plasma column bubbler, no ammonia could be detected in the electrolyte, showing the specificity of the preferred embodiments of the invention to be specific toward the production of NOₓ. In the case of the electrolysis of PAW, the significant production rate of 45 nmol cm⁻² s⁻¹ was obtained.

Moreover, the measured concentration of ammonia in the background electrolyte, as well as all of the other controls, was more than four orders of magnitudes lower than the measured ammonia in the electrolysis tests. This result indicates that the environmental contaminations are not contributing to the ammonia production rate reported in this work.

### Build Up Of NOX Species Over Time

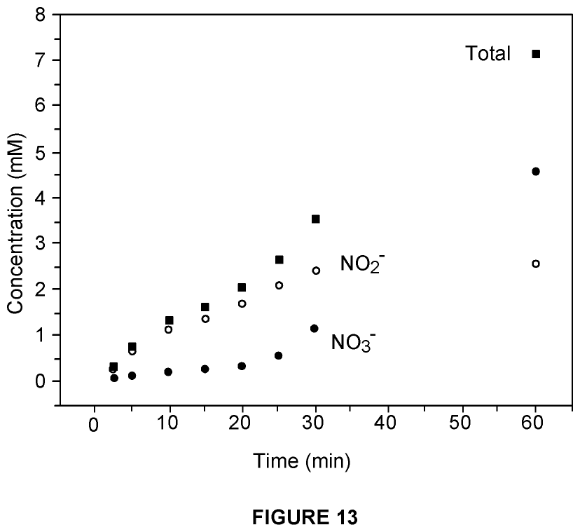

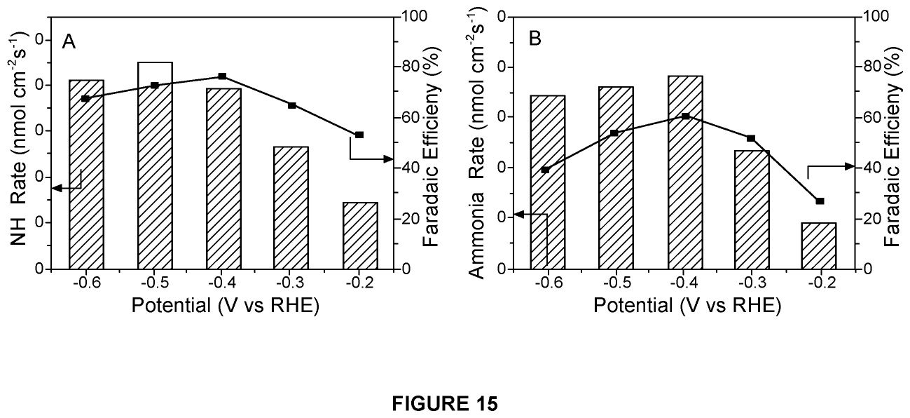

The concentration of NOₓ was controlled by the plasma activation time under the optimized parameters (voltage of 100V, duty of 83 μs, discharge frequency of 600 Hz and resonance frequency of 60 Hz). It is shown from FIG. 13, the total amount of NOₓ increases linearly as a function of plasma activation time. In this study, 0.5 h of plasma activation (produces ˜4 mM NOₓ in 100 mL of water) was selected for the electrolysis tests. This plasma activation time was selected based on the systematic study on the effect of NOₓ concentration (using nitrate and nitrite salts) on the FE and production rate of ammonia (FIG. 15).

### Electrochemical Optimization

The first step toward the optimization of the electrocatalytic conversion of NOₓ to ammonia was performed using nitrate (KNO₃) and nitrite (NaNO₂) salts as the NOₓ source, and Cu foil (1 cm×1 cm) as the cathode, Pt wire as the anode and Ag/AgCl (sat. KCl) as the reference electrode in a custom-designed H-cell (FIG. 5B).

In acidic media, the reaction proceeds, as shown in Equations S1-S2 below. The reaction competes with the hydrogen evolution reaction (HER), Equation S3[1].

| | | |
|---|---|---|
| NO₃⁻ + 10H⁺ + 8e⁻ → NH₄⁺ + 3H₂0 | E⁰ = 0.93 V vs RHE | (Eq. S1) |
| NO₂⁻ + 8H⁺ + 6e⁻ → NH₄⁺ + 2H₂O | E⁰ = 0.86 V vs RHE | (Eq. S2) |
| 2H⁺ + 2e⁻ → H₂ | E⁰ = 0 V vs RHE | (Eq. S3) |

Whilst H⁺ is required to facilitate the reaction, a high concentration of H⁺ may result in occurrence of HER. In the present experiment, when H⁺ was not available in the electrolyte, both FE and ammonia yield rate was very low (>30% and 1 nm cm⁻²s⁻¹, respectively). The addition of acid (10 mM H₂SO₄) in the electrolyte resulted in a significant increase in the ammonia production rate and FE, from 0.81 to 8.94 nmolcm⁻² s⁻¹ and from 31% to 73%, respectively. However, a further increase in the acid concentration did not positively impact the ammonia production while the FE dropped significantly as

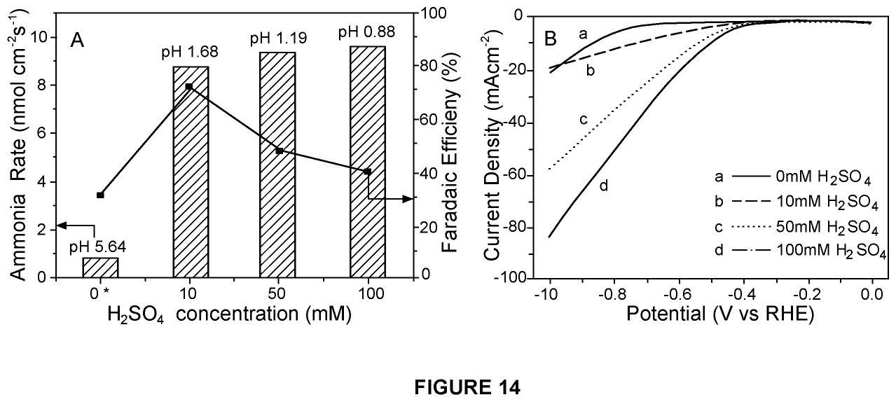

HER became more competitive. See FIG. 14.

Therefore, in this study, 10 mM H₂SO₄ was used as the background electrolyte which (a) increases the conductivity of the electrochemical system to minimize energy losses caused by the resistance; (b) provides proton for ammonia synthesis; (c) supports synthesis of ammonium sulphate (NH₄)₂SO₄ which is soluble in water and can be used directly as a fertilizer. See FIG. 15

When nitrite was used as the reactant, ammonia production rate increased with more negative potentials, and the rate reaches its maximum at −0.5V with FE of about 73%. While for nitrate, its maximum production rate (˜3.8 nmol cm⁻² s⁻¹) and FE (˜60%) occurred at −0.4V. Beyond the optimal potential, both rate and FE start to decrease due to a possible occurrence of HER.

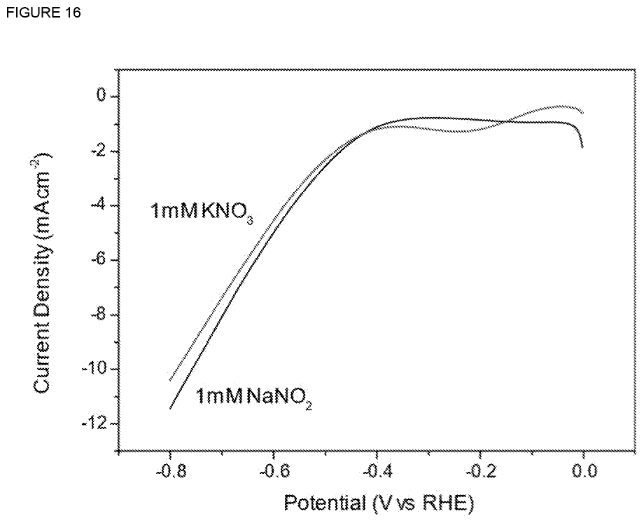

FIG. 16 compares the LSV curves of nitrate and nitrite salts in 10 mM H₂SO₄. The reduction of nitrate to nitrite (Eq. S4) is evidenced by a peak occurred at around −0.25V on the LSV curve of the nitrate solution (1 mM KNO₃).

NO₃⁻30 2H⁺+2e⁻→NO₂⁻+H₂O (Eq. S4)

This result indicates that at lower potentials, nitrate is more favourable to be converted to nitrite rather than ammonia (which is in agreement with the literature [2]). To investigate, a 15 min electrolysis experiment was conducted in 25 mL of 1 mM KNO₃ solution at −0.3V. It was found that 0.7 μmol of ammonia was produced, while 1.17 μmol of nitrite was produced in this potential. On the other hand, when the experiment was performed at −0.5V, 1.64 μmol ammonia and 0.66 μmol nitrite were produced.

During NOₓ reduction, it established that NO₃_{^{−}} is first adsorbed to the surface of the electrode to form *NO₃ and the N—O bond is then spontaneously cleaved stepwise producing *NO₂ and *NO. Next, the hydrogenation of *NO to form *NOH occurs. Successively, the *NOH hydrogenated to form *NH₂OH and then transformed into *NH₃. Finally, *NH₃ desorbed from the catalyst. In this process it was observed that a portion of the *NO₂ desorbed into the solution as NO₂_{^{−}} at lower potentials. This clarifies the reason behind the observed lower FE of ammonia conversion at the lower potentials. At higher potentials, however, the conversion rate of both nitrate and nitrite to ammonia is very high, which compensates for this side reaction.

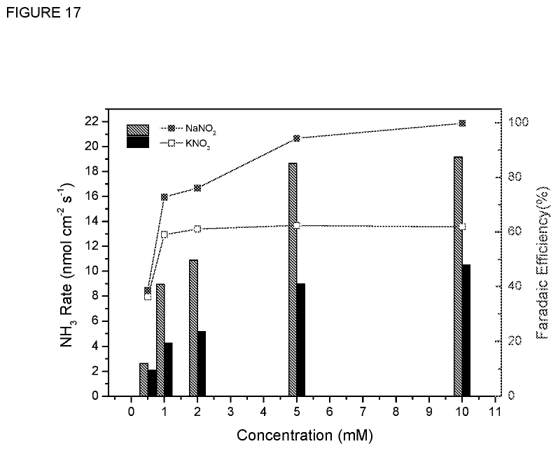

To investigate the effect of NOₓ concentration on the ammonia production rate and FE, a range of concentrations of nitrite and nitrate salts was tested (FIG. 17). In the case of NO₂_{^{−}} salt, much higher production rate and FE were observed compared to NO₃_{^{−}} salt. Please note, the FE of the conversion of NO₂_{^{−}} to ammonia can reach 100% while the FE of NO₃_{^{−}} stays at around 60%. The lower FE when NO₃_{^{−}} was used can be ascribed to some charge lose arising from the conversion to NO₃_{^{−}} to NO₂_{^{−}} species instead of ammonia. However, both NO₃_{^{−}} and NO₂_{^{−}} are converted to ammonia in the end. For both nitrite and nitrate, ammonia production rates and FE increased significantly when the concentration reached at ˜1 mM. This graph was a guide to set the duration of the plasma activation (achieving NOₓ concentration of >1 mM) to maximize the ammonia production rate and FE.

Effect of pH Upon Electrolysis

A comparative study was carried out in which a series of electrocatalytic reduction reactions were carried out under identical conditions, save for varying the starting pH. Table 3 displays the corresponding experimental conditions.

TABLE 3
Conditions of ammonia production rate data results
Experiment Condition

| | | |
|---|---|---|
| Electrode | Working | Cu/Cu₂O NWs on Cu Foam, 1 cm² |
| | | (electro-reduced from CuO NWs ) |
| | Counter | Pt |
| | Reference | Ag/AgCl |
| Electrolyte | pH | 1.68 (no acid added, low pH was |
| | | caused by plasma) |
| | KCl concentration | 50 mM |
| Electrolysis | Electrolyte Volume | 25 mL |
| | Time | 15 min |
| | Applied Potentials | from −0.3 V to −0.7 V vs RHE |
| | Stirring Speed | 650 rpm |

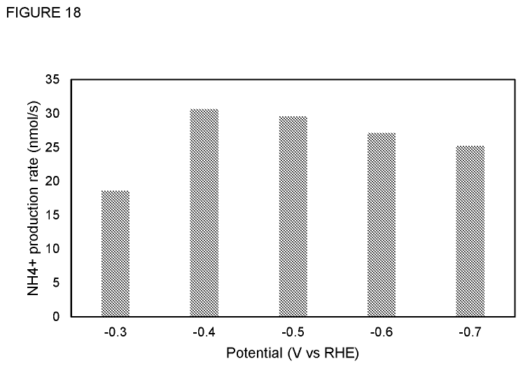

Analysis was undertaken with a range of pH conditions. For example, plasma activated water (PAW), salt (KCl) with a concentration of 50 mM was added to PAW, allowing the electrolysis to occur in a neutral media. From this and other experiments, the present inventors have concluded that the reduction method of the present invention may be carried out at any pH. FIG. 18 displays the ammonia production rate of the system.

### Catalyst Surface Area Effect

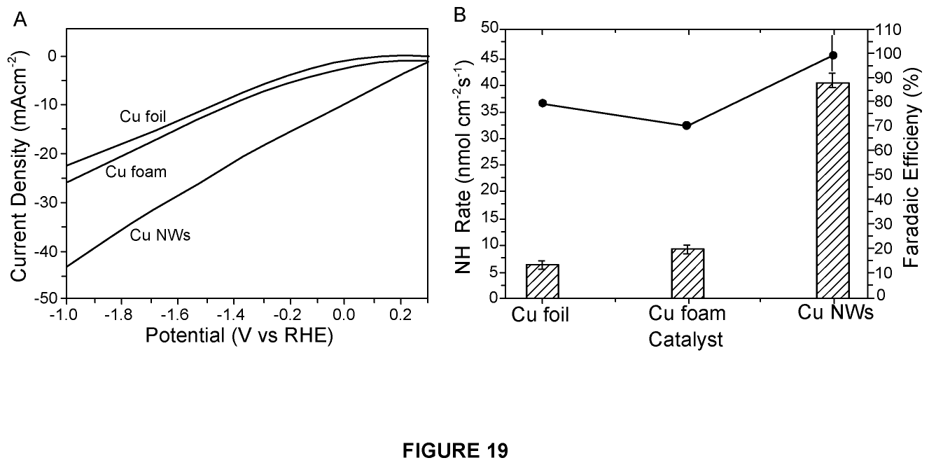

To compare copper-based catalyst performance as a function of available surface area, control experiments were performed with Cu foil and Cu foam. Cu NWs electrode facilitated a very high current density (j), −45 mA cm⁻² at −1V, compared to −22 mA cm⁻² for Cu foil and −26 mA cm⁻² for Cu foam. Cu NWs also facilitated a much higher catalytic activity for ammonia synthesis with a production rate of 40±3.3 nmol cm⁻² s⁻¹ and FE of 100±7%. At the same time, Cu foil only provided ammonia production rate of 6.1±0.6 nmol cm⁻² s⁻¹ with FE of 80.6±0.3% and Cu foam had a rate of 8.8±1.3 nmol cm⁻² s⁻¹ with FE of 71.1±1.7%. See FIG. 19

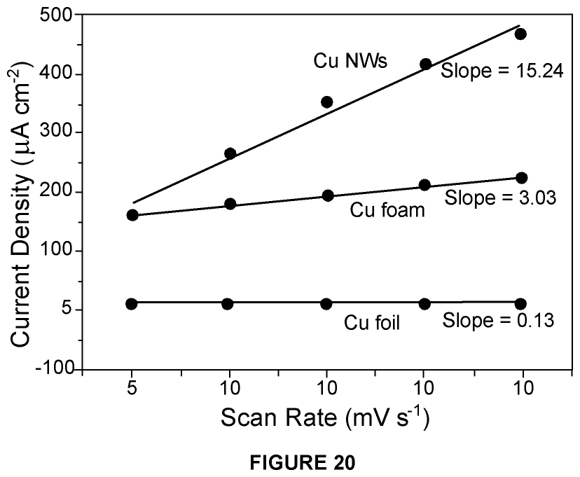

In the case of Cu foil and Cu foam, non-Faradaic charging currents are measured in the potential range of 0.5V and 0.55V vs RHE and for Cu NWs, the potential range is 0.25V to 0.30V vs RHE. The scan rate is varied between 5, 10, 15, 20 and 25 mV/s and the anodic (positive) and cathodic (negative) current densities are obtained from the double layer charge/discharge curves at 0.525V vs RHE for Cu foil and Cu foam and 0.275V vs RHE for Cu NWs. See FIG. 20.

The double-layer capacitance was then calculated by averaging the absolute values of cathodic and anodic current densities and take the slopes of the linear fits. The slopes obtained with Cu foil, Cu foam and Cu NWs are 0.13 mF/cm², 3.03 mF/cm² and 15.24 mF/cm² respectively, indicating that the fabricated catalyst Cu NWs has much larger electrochemical active surface area compared to the commercial Cu foil and Cu foam.

### Catalyst Species

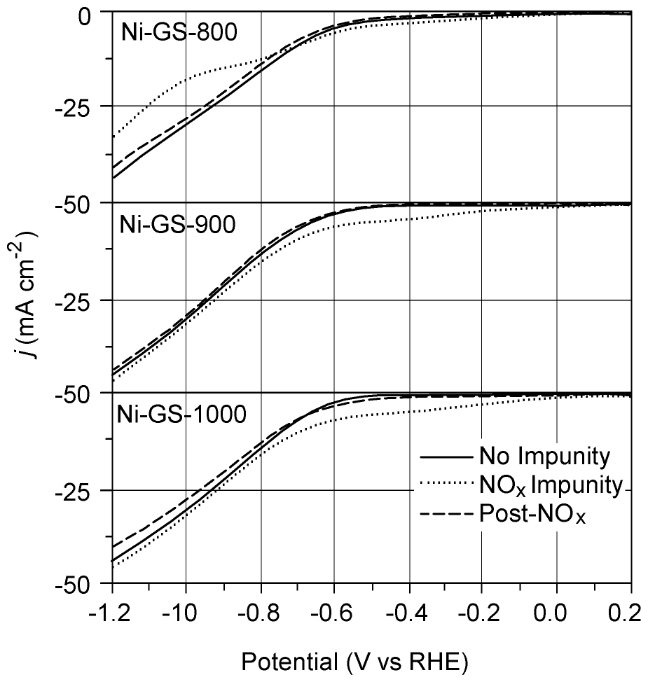

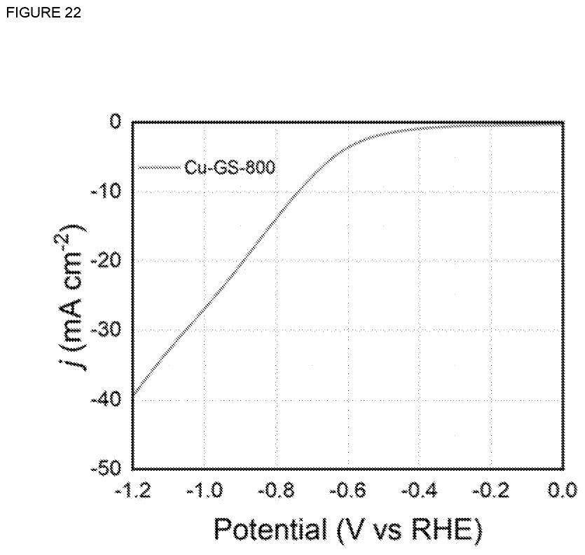

The performance of a nickel based and single-atom copper catalysts in the electrocatalytic reduction of nitrate to ammonia were investigated and the results are shown in FIGS. 21 and 22. These results confirmed the efficacy of different transition metals, and catalyst types in the electrocatalytic reduction of the present invention

### Reduction of Nox Species Over Time

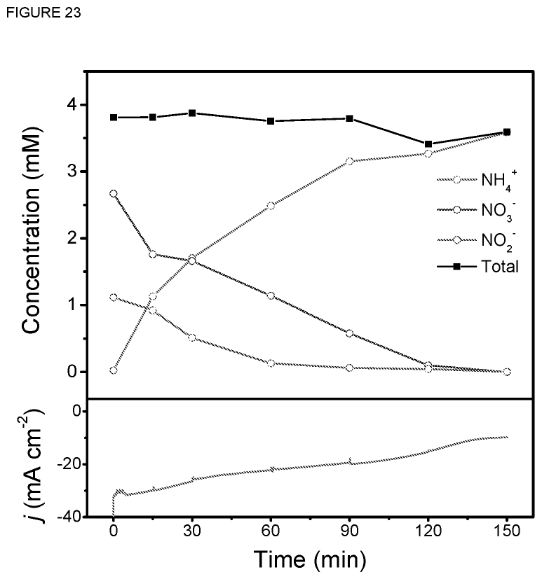

With an extended reaction time of 2.5 h (See FIG. 23), both NO₃_{^{−}} and NO₂_{^{−}} species were completely exhausted (from 2.7 mM and 1 mM, respectively). On the other hand, ammonia concentration increases from 0 mM to 3.5 mM over the same period. A slightly lower final concentration of ammonia compared to initial concentration of NOₓ was obtained (3.5 vs 3.7 mM). This reduction can be attributed to the loss of a small amount of NOx and ammonia due to sampling. The chronoamperometric i-t curve displays a consistently declining j which indicates the consumption of the reactants during this 2.5 h.

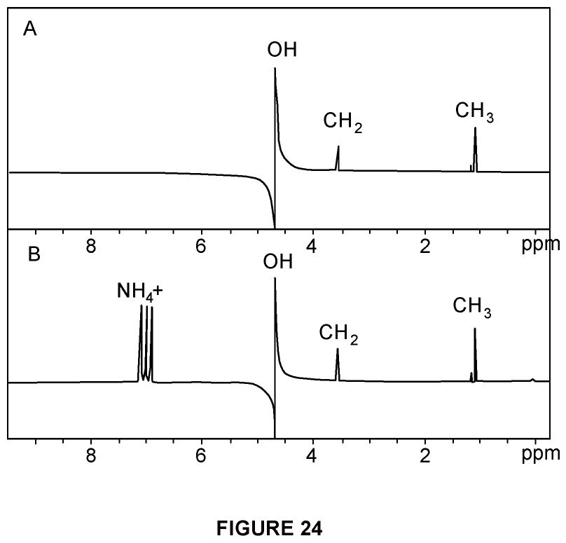

NMR analysis (see FIG. 24) also supports the formation of ammonia with non-detection amount in the PAW solution before electrocatalysis. In general, the peak for N₂H₄ occurs at the chemical shift of 3.2 ppm and the result indicates that there is no N₂H₄ in the final solution. There are two other small peaks are ascribed to —CH₃ and —CH₂ respectively, (—OH is the main peak with water as the background solution), which is caused by the impurity of ethanol.

### CO₂ Reduction

A similar series of experiments was conducted to those described above, with the input gas changed from air to carbon dioxide reduction, in order to investigate carbon dioxide reduction.

Plasma runs were conducted for 10 minutes by continuously bubbling CO₂ gas through Milli-Q water at 0.1-0.5 L min⁻¹. For these tests, Plasma Leap was operated at voltage of 200 V, duty cycle of 83 μs, discharge frequency of 2 kHz and resonance frequency of 60 kHz. Subsequently, activated species in liquid phase were electrochemically converted to hydrocarbon products. Two different catalysts, i.e. Cu foam and Ni, were selected as cathodes. While CO₂ to CO conversion was prevalent, some higher hydrocarbons produced. The tests evidenced that the invention can be successfully utilized for CO₂ conversion to high value chemicals.

### Techno-Economic Calculations

The global $60 billion ammonia fertiliser market is supplied by ammonia generated using the conventional Haber-Bosch process at an average price of $0.23-25 per kg. Locally, the price ranges from $0.2-$0.5 per kg of ammonia. Due to the advantages experienced by large plants due to economies of scale, almost all fertilizer plants are large-scale (˜100,000 MT per year), located strategically near ports for water requirement as well as for shipping and as a result, a significant infrastructure is required to transport fertilizer to rural farms and locations. Hence, local farmers are required to pay a significantly higher price, i.e. 5 kg of ammonia fertilizer costs $10.58. (AUD as at September 2020)

As such, considerable efforts are in place to generate ammonia in small-scale delocalized units at a competitive cost. While electrochemical nitrogen reduction reactions to ammonia are proposed as a promising technology, the best performing NRR catalyst can generate ammonia with a low yield of merely 0.23 μmol h⁻¹ cm⁻² at RTP, with a high energy input of 1410 kWh/kg_{NH3}. It must be stated that in addition to the high cost, the overall yield within these electrochemical NRR are very low, making these systems unfavourable for scalability.

In contrast, the hybrid NRR system of the present invention is capable of generating ammonia with a yield which is ˜3,000 times greater than the NRR counterpart. As indicated in FIG. 5, using a pin-nozzle plasma design and in a H-cell, the hybrid system of the present invention is capable of generating ammonia with the pin-to-liquid bubbler column plasma system generating NOx at 3.8 kWh/mol, which is at least three times more energy-efficient than state of the art. The flow-through electrolyzer can produce ammonia directly with specific energy consumption as low as 0.19 kWh/mol ammonia.

1. A method of reducing a gaseous compound comprising the steps of:
subjecting the gaseous compound to plasma forming conditions to form a plasma;
contacting the plasma with water or an electrolyte at a plasma-water or electrolyte-water interface, thereby to provide a dissolved plasma derived species; and
electrocatalytically reducing said dissolved plasma derived species to provide a reduced compound.

2. A method according to claim 1 wherein the gaseous compound is an oxygen containing compound or is mixed with oxygen.

3. A method according to claim 1 wherein the gaseous compound is carbon dioxide.

4. A method according to claim 1 wherein the gaseous compound is nitrogen mixed with oxygen.

5. A method according to claim 1, wherein the plasma is generated by a combination of glow discharge and spark discharge in a configuration of a pin-to-liquid with no enclosure, pin-to-liquid with nozzle enclosure, or a pin-to-liquid with a column bubbler enclosure.

6. A method according to claim 1 wherein the plasma-water or electrolyte water interface is at the interface of a bubble of gas in the water or electrolyte.

7. A method according to claim 1 wherein the gaseous compound is provided at controlled humidity and/or a controlled temperature and/or a controlled pH.

8-13. (canceled)

14. A method of reducing nitrogen containing gas to produce ammonia, the method comprising the steps of:
subjecting the nitrogen containing gas to plasma forming conditions to form a nitrogen containing plasma;
contacting the nitrogen containing plasma with water or an electrolyte at a plasma-water or electrolyte-water interface, thereby to provide dissolved NOₓ species; and
electrocatalytically reducing said NOₓ to provide ammonia.

15. A method according to claim 14 wherein the nitrogen containing gas is selected from the group consisting of N₂; a nitrogen gas further comprising oxygen; and air.

16-18. (canceled)

19. A method according to claim 14 wherein the plasma is generated by a combination of glow discharge and spark discharge or where the plasma is generated by pin discharge.

20. A method according to claim 14 wherein the plasma-water or electrolyte water interface is at the interface of a bubble of gas in the water or electrolyte.

21-22. (canceled)

23. A method according to claim 14 wherein the electrolyte is an aqueous electrolyte.

24-26. (canceled)

27. A method according to claim 14 wherein the dissolved NOₓ species are NO₂_{^{−}} or NO₃_{^{−}}.

28. (canceled)

29. A method according to claim 1 wherein the electrocatalytic reduction is facilitated by a transition metal catalyst or transition metal oxide catalyst.

30-31. (canceled)

32. A method according to claim 29 wherein the transition metal catalyst is located in the reaction system in a region adjacent the region of the spark discharge and/or glow discharge

33. A method of reducing carbon containing gas comprising the steps of:
subjecting the carbon containing gas to plasma forming conditions to form a carbon plasma;
contacting the carbon plasma with water or an electrolyte at a plasma-water or electrolyte-water interface, thereby to provide a dissolved COₓ species; and
electrocatalytically reducing said COₓ species to provide one or more reduced compounds selected from CO, syngas or formate.

34. A method according to claim 33 wherein the carbon containing gas is i) an oxygen containing species or ii) further comprises O₂.

35. A method according to claim 33 wherein the carbon containing gas is carbon dioxide.

36. Apparatus for reducing a gas comprising:
i) a feed line to feed the gas to a glow/spark plasma discharge (pin-to-liquid with no enclosure, pin-to-liquid with nozzle enclosure, or a pin-to-liquid with a column bubbler enclosure) located at or below a liquid level in a reaction vessel;
ii) the pin-to-liquid with no enclosure, pin-to-liquid with nozzle enclosure, or a pin-to-liquid with a column bubbler enclosure having a housing configured to generate bubbles in the liquid in a reaction vessel when in use; and
iii) a feed line to transport dissolved plasma species from the reaction vessel to an electrocatalytic reduction chamber.

37. Apparatus for reducing a gas comprising
i) a feed line to feed the gas to a glow/spark plasma discharge (pin-to-liquid with no enclosure, pin-to-liquid with nozzle enclosure, or a pin-to-liquid with a column bubbler enclosure) located at or below a liquid level in a reaction vessel;
ii) the pin-to-liquid with no enclosure, pin-to-liquid with nozzle enclosure, or a pin-to-liquid with a column bubbler enclosure having a housing configured to generate bubbles in the liquid in a reaction vessel when in use;
iii) a fluid line to transport dissolved plasma reaction products from the reaction vessel to a reservoir; and
iv) a feed line to transport dissolved plasma species from the reservoir to an electrocatalytic reduction chamber.

38-41. (canceled)
